(19) United States
(12) Patent Application Publication
(10) Pub. No.: US 2011/0288417 A1
(43) Pub. Date: Nov. 24, 2011

(54) MOBILE VIDEOCONFERENCING ROBOT SYSTEM WITH AUTONOMY AND IMAGE ANALYSIS

(75) Inventors: Marco Pinter, Santa Barbara, CA (US); Fuji Lai, Goleta, CA (US); Yulun Wang, Goleta, CA (US); H. Neal Reynolds, Severna Park, MD (US)

(73) Assignee: INTOUCH TECHNOLOGIES, INC., Goleta, CA (US)

(21) Appl. No.: 13/111,208

(22) Filed: May 19, 2011

Related U.S. Application Data
(60) Provisional application No. 61/346,405, filed on May 19, 2010.

(51) Int. Cl. A61B 6/00; A61B 5/11; B25J 13/08

(52) U.S. Cl. 600/473; 700/259; 600/595; 901/1; 901/47

(57) ABSTRACT

A robot system that can move about two or more patient beds. The robot includes a monitor and an infrared camera that are coupled to a mobile platform. The robot also includes a controller that is programmed to autonomously move the mobile platform from one patient to another patient and process images captured by the infrared camera to determine if one or more of the patients needs assistance. By way of example, the robot can determine whether a patient is out of a bed, or in a position wherein they may fall out of the bed. The robot may be coupled to a remote station that allows an operator to move the robot and conduct a videoconference with the patient. The image captured by the infrared robot camera can be utilized to analyze blood flow of the patient. The robot can also be utilized to perform neurological analysis.

### REFERENCE TO CROSS-RELATED APPLICATION

This application claims priority to U.S. Provisional Patent Application No. 61/346,405, titled ENHANCED VISUALIZATION AND AUTONOMY FOR TELEHEALTH ROBOTS TO ADDRESS HEALTHCARE AVAILABILITY, filed on May 19, 2010, the entire contents of which are hereby incorporated by reference herein.

### BACKGROUND

1. Field of the Invention

The subject matter disclosed generally relates to the field of mobile two-way teleconferencing.

2. Background Information

Robots have been used in a variety of applications ranging from remote control of hazardous material to assisting in the performance of surgery. For example, U.S. Pat. No. 5,762,458 issued to Wang et al. discloses a system that allows a surgeon to perform minimally invasive medical procedures through the use of robotically controlled instruments. One of the robotic arms in the Wang system moves an endoscope that has a camera. The camera allows a surgeon to view a surgical area of a patient.

There has been marketed a mobile robot introduced by InTouch Technologies, Inc., the assignee of this application, under the trademarks COMPANION and RP-7. The InTouch robot is controlled by a user at a remote station. The remote station may be a personal computer with a joystick that allows the user to remotely control the movement of the robot. Both the robot and the remote station have cameras, monitors, speakers and microphones to allow for two-way video/audio communication. The robot camera provides video images to a screen at the remote station so that the user can view the robot's surroundings and move the robot accordingly.

U.S. Pat. No. 7,142,947 issued to Wang, et al. and assigned to InTouch Technologies, Inc. discloses use of robots to monitor patients. A doctor at a remote station can move the robot about a healthcare facility to view a patient. U.S. Pat. No. 5,802,494 issued to Kuno also discloses the use of a robot to view a patient within a room. The Kuno robot includes an infrared camera that allows for night viewing. Kuno discloses a process wherein the patient's facial expression is analyzed to determine whether they need medical attention.

### BRIEF DESCRIPTION OF THE DRAWINGS

FIG. 1 is an illustration of a robotic system;

FIG. 2 is a schematic of an electrical system of a robot;

FIG. 3 is a further schematic of the electrical system of the robot; and,

FIG. 4 is a graphical user interface of a remote station.

### DETAILED DESCRIPTION

Disclosed is a robot system that can move about two or more patient beds. The robot includes a monitor and an infrared camera that are coupled to a mobile platform. The robot also includes a controller that is programmed to autonomously move the mobile platform from one patient to another patient and process images captured by the infrared camera to determine if one or more of the patients needs assistance. By way of example, the robot can determine whether a patient is out of a bed, or in a position wherein they may fall out of the bed. The robot may be coupled to a remote station that allows an operator to move the robot and conduct a videoconference with the patient. The image captured by the infrared robot camera can be utilized to analyze blood flow of the patient. The robot can also be utilized to perform neurological analysis.

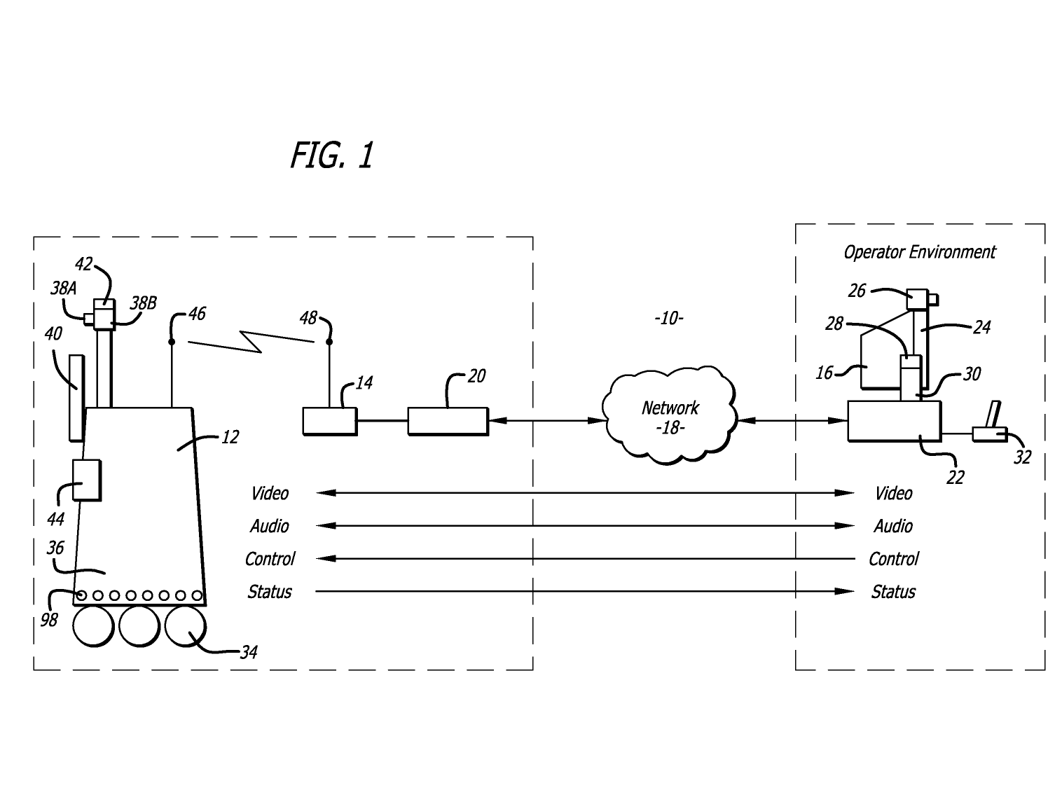

Referring to the drawings more particularly by reference numbers, FIG. 1 shows a robotic system 10 that can be used to conduct a remote visit. The robotic system 10 includes a robot 12, a base station 14 and a remote control station 16. The remote control station 16 may be coupled to the base station 14 through a network 18. By way of example, the network 18 may be either a packet switched network such as the Internet, or a circuit switched network such has a Public Switched Telephone Network (PSTN) or other broadband system. The base station 14 may be coupled to the network 18 by a modem 20 or other broadband network interface device. By way of example, the base station 14 may be a wireless router. Alternatively, the robot 12 may have a direct connection to the network thru for example a satellite.

The remote control station 16 may include a computer 22 that has a monitor 24, a camera 26, a microphone 28 and a speaker 30. The computer 22 may also contain an input device 32 such as a joystick or a mouse. The control station 16 is typically located in a place that is remote from the robot 12. Although only one remote control station 16 is shown, the system 10 may include a plurality of remote stations. In general any number of robots 12 may be controlled by any number of remote stations 16 or other robots 12. For example, one remote station 16 may be coupled to a plurality of robots 12, or one robot 12 may be coupled to a plurality of remote stations 16, or a plurality of robots 12.

Each robot 12 includes a movement platform 34 that is attached to a robot housing 36. Also attached to the robot housing 36 is a pair of cameras 38A and 38B, a monitor 40, a microphone(s) 42 and a speaker(s) 44. The camera 38B is an infrared device that can view objects such as a patient at night. The microphone 42 and speaker 30 may create a stereophonic sound. The robot 12 may also have an antenna 46 that is wirelessly coupled to an antenna 48 of the base station 14. The system 10 allows a user at the remote control station 16 to move the robot 12 through operation of the input device 32. The robot cameras 38A and 38B are coupled to the remote monitor 24 so that a user at the remote station 16 can view a patient. Likewise, the robot monitor 40 is coupled to the remote camera 26 so that the patient can view the user. The microphones 28 and 42, and speakers 30 and 44, allow for audible communication between the patient and the user.

The remote station computer 22 may operate Microsoft OS software and WINDOWS XP or other operating systems such as LINUX. The remote computer 22 may also operate a video driver, a camera driver, an audio driver and a joystick driver. The video images may be transmitted and received with compression software such as MPEG CODEC.

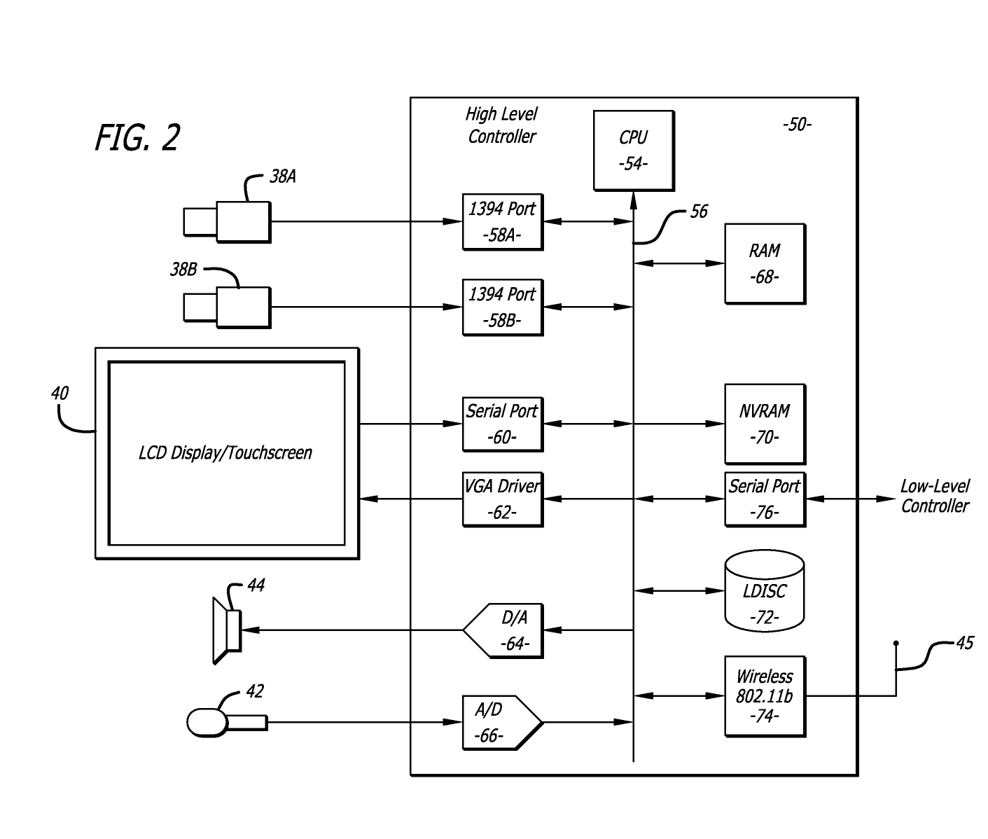

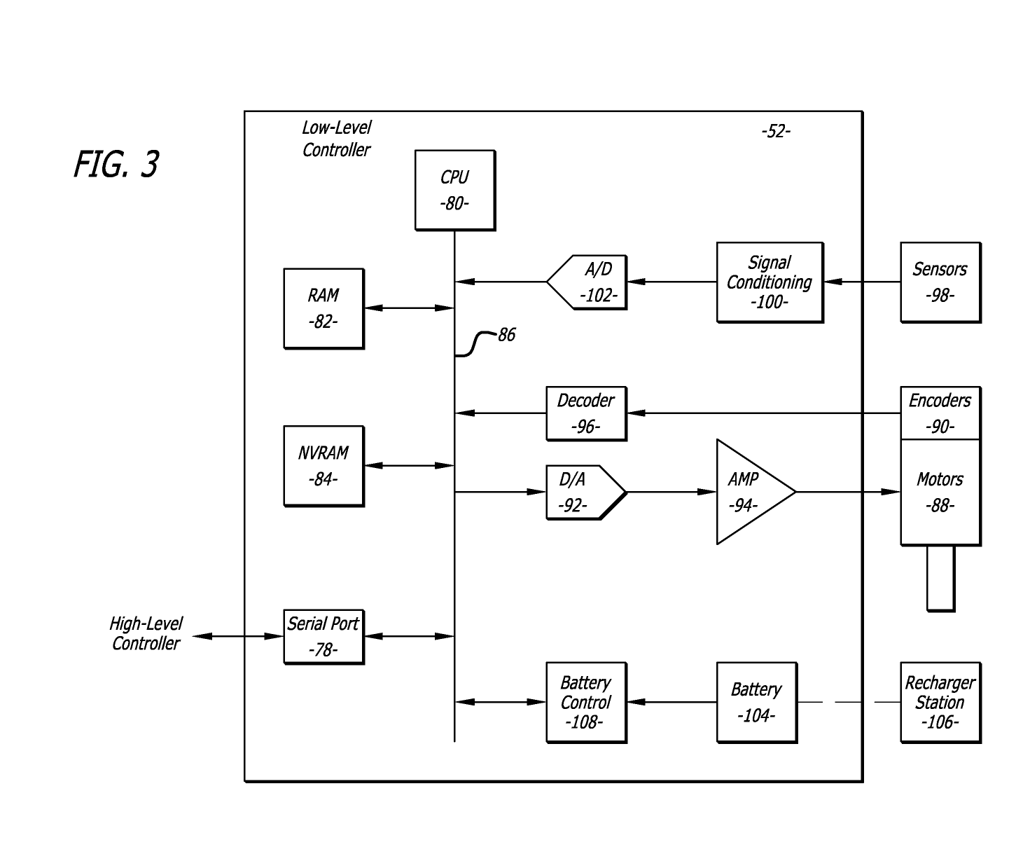

FIGS. 2 and 3 show an embodiment of a robot 12. Each robot 12 may include a high level control system 50 and a low level control system 52. The high level control system 50 may include a processor 54 that is connected to a bus 56. The bus 56 is coupled to the cameras 38A and 38B by an input/output (I/O) ports 58A and 58B, respectively. The monitor 40 is coupled to the bus 56 by a serial output port 60 and a VGA driver 62. The monitor 40 may include a touchscreen function that allows the patient to enter input by touching the monitor screen.

The speaker 44 is coupled to the bus 56 by a digital to analog converter 64. The microphone 42 is coupled to the bus 56 by an analog to digital converter 66. The high level controller 50 may also contain random access memory (RAM) device 68, a non-volatile RAM device 70 and a mass storage device 72 that are all coupled to the bus 56. The mass storage device 72 may contain medical files of the patient that can be accessed by the user at the remote control station 16. For example, the mass storage device 72 may contain a picture of the patient. The user, particularly a health care provider, can recall the old picture and make a side by side comparison on the monitor 24 with a present video image of the patient provided by the cameras 38A and 38B. The robot antennae 46 may be coupled to a wireless transceiver 74. By way of example, the transceiver 74 may transmit and receive information in accordance with IEEE 802.11b.

The controller 54 may operate with a LINUX OS operating system. The controller 54 may also operate MS WINDOWS along with video, camera and audio drivers for communication with the remote control station 16. Video information may be transceived using MPEG CODEC compression techniques. The software may allow the user to send e-mail to the patient and vice versa, or allow the patient to access the Internet. In general the high level controller 50 operates to control communication between the robot 12 and the remote control station 16.

The remote control station 16 may include a computer that is similar to the high level controller 50. The computer would have a processor, memory, I/O, software, firmware, etc. for generating, transmitting, receiving and processing information.

The high level controller 50 may be linked to the low level controller 52 by serial ports 76 and 78. The low level controller 52 includes a processor 80 that is coupled to a RAM device 82 and non-volatile RAM device 84 by a bus 86. Each robot 12 contains a plurality of motors 88 and motor encoders 90. The motors 88 can actuate the movement platform and move other parts of the robot such as the monitor and camera. The encoders 90 provide feedback information regarding the output of the motors 88. The motors 88 can be coupled to the bus 86 by a digital to analog converter 92 and a driver amplifier 94. The encoders 90 can be coupled to the bus 86 by a decoder 96. Each robot 12 also has a number of proximity sensors 98 (see also FIG. 1). The position sensors 98 can be coupled to the bus 86 by a signal conditioning circuit 100 and an analog to digital converter 102.

The low level controller 52 runs software routines that mechanically actuate the robot 12. For example, the low level controller 52 provides instructions to actuate the movement platform to move the robot 12. The low level controller 52 may receive movement instructions from the high level controller 50. The movement instructions may be received as movement commands from the remote control station or another robot. Although two controllers are shown, it is to be understood that each robot 12 may have one controller, or more than two controllers, controlling the high and low level functions.

The various electrical devices of each robot 12 may be powered by a battery(ies) 104. The battery 104 may be recharged by a battery recharger station 106. The low level controller 52 may include a battery control circuit 108 that senses the power level of the battery 104. The low level controller 52 can sense when the power falls below a threshold and then send a message to the high level controller 50.

The system may be the same or similar to a robotic system provided by the assignee InTouch-Health, Inc. of Santa Barbara, Calif. under the name RP-7. The system may also be the same or similar to the system disclosed in U.S. Pat. No. 6,925,357 issued Aug. 2, 2005, which is hereby incorporated by reference.

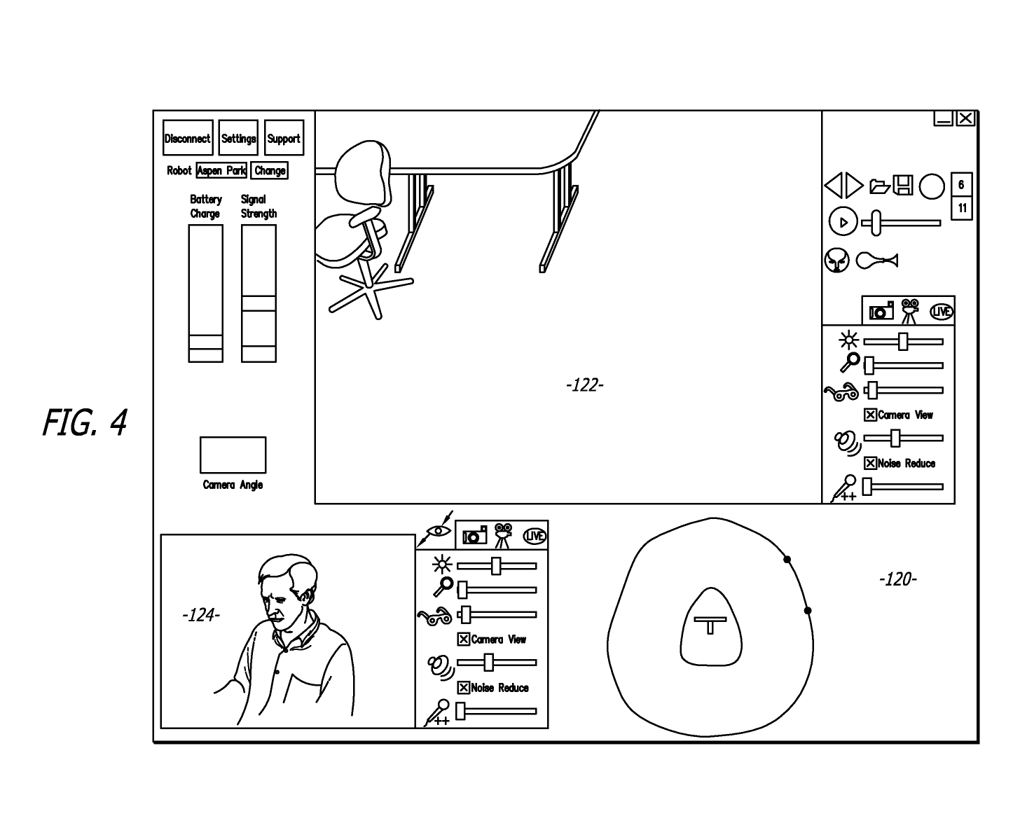

FIG. 4 shows a display user interface (“DUI”) 120 that can be displayed at the remote station 16. The DUI 120 may include a robot view field 122 that displays a video image provided by the camera of the robot. The DUI 120 may also include a station view field 124 that displays a video image provided by the camera of the remote station 16. The DUI 120 may be part of an application program stored and operated by the computer 22 of the remote station 16.

In operation, the robot 12 may be placed in a home or a facility where one or more patients are to be monitored and/or assisted. The facility may be a hospital or a residential care facility. By way of example, the robot 12 may be placed in a home where a health care provider may monitor and/or assist the patient. Likewise, a friend or family member may communicate with the patient. The cameras and monitors at both the robot and remote control stations allow for teleconferencing between the patient and the person at the remote station(s).

The robot 12 can be maneuvered through the home or a facility by manipulating the input device 32 at a remote station 16. The robot 12 may be controlled by a number of different users. To accommodate for this the robot may have an arbitration system. The arbitration system may be integrated into the operating system of the robot 12. For example, the arbitration technique may be embedded into the operating system of the high-level controller 50.

By way of example, the users may be divided into classes that include the robot itself, a local user, a caregiver, a doctor, a family member, or a service provider. The robot 12 may override input commands that conflict with robot operation. For example, if the robot runs into a wall, the system may ignore all additional commands to continue in the direction of the wall. A local user is a person who is physically present with the robot. The robot could have an input device that allows local operation. For example, the robot may incorporate a voice recognition system that receives and interprets audible commands.

A caregiver is someone who remotely monitors the patient. A doctor is a medical professional who can remotely control the robot and also access medical files contained in the robot memory. The family and service users remotely access the robot. The service user may service the system such as by upgrading software, or setting operational parameters.

The robot 12 may operate in one of two different modes; an exclusive mode, or a sharing mode. In the exclusive mode only one user has access control of the robot. The exclusive mode may have a priority assigned to each type of user. By way of example, the priority may be in order of local, doctor, caregiver, family and then service user. In the sharing mode two or more users may share access with the robot. For example, a caregiver may have access to the robot, the caregiver may then enter the sharing mode to allow a doctor to also access the robot. Both the caregiver and the doctor can conduct a simultaneous tele-conference with the patient.

The arbitration scheme may have one of four mechanisms; notification, timeouts, queue and call back. The notification mechanism may inform either a present user or a requesting user that another user has, or wants, access to the robot. The timeout mechanism gives certain types of users a prescribed amount of time to finish access to the robot. The queue mechanism is an orderly waiting list for access to the robot. The call back mechanism informs a user that the robot can be accessed. By way of example, a family user may receive an e-mail message that the robot is free for usage. Tables I and II, show how the mechanisms resolve access request from the various users.

TABLE I

| | Access | Medical | Command | Software/Debug | Set |
|---|---|---|---|---|---|
| User | Control | Record | Override | Access | Priority |
| Robot | No | No | Yes (1) | No | No |
| Local | No | No | Yes (2) | No | No |
| Caregiver | Yes | Yes | Yes (3) | No | No |
| Doctor | No | Yes | No | No | No |
| Family | No | No | No | No | No |
| Service | Yes | No | Yes | Yes | Yes |

TABLE II
Requesting User

| | Local | | Caregiver | Doctor | Family | Service |
|---|---|---|---|---|---|---|
| Current User | Local | Not Allowed | Warn current user | Warn current user | Warn current user | Warn current user |
| | | | of pending user | of pending user | of pending user | of pending user |
| | | | Notify requesting | Notify requesting | Notify requesting | Notify requesting |
| | | | user that system is | user that system is | user that system is | user that system is |
| | | | in use | in use | in use | in use |
| | | | Set timeout | Set timeout = 5 m | Set timeout = 5 m | No timeout |
| | | | | | Call back | Call back |
| | Caregiver | Warn current | Not allowed | Warn current user | Warn current user | Warn current user |
| | | user of pending | | of pending user | of pending user | of pending user |
| | | user. | | Notify requesting | Notify requesting | Notify requesting |
| | | Notify | | user that system is | user that system is | user that system is |
| | | requesting user | | in use | in use | in use |
| | | that system is | | Set timeout = 5 m | Set timeout = 5 m | No timeout |
| | | in use. | | Queue or callback | | Callback |
| | | Release | | | | |
| | | control | | | | |
| | Doctor | Warn current | Warn current user | Warn current user | Notify requesting | Warn current user |
| | | user of pending | of pending user | of pending user | user that system is | of pending user |
| | | user | Notify requesting | Notify requesting | in use | Notify requesting |
| | | Notify | user that system is | user that system is | No timeout | user that system is |
| | | requesting user | in use | in use | Queue or callback | in use |
| | | that system is | Set timeout = 5 m | No timeout | | No timeout |
| | | in use | | Callback | | Callback |
| | | Release | | | | |
| | | control | | | | |
| | Family | Warn current | Notify requesting | Warn current user | Warn current user | Warn current user |
| | | user of pending | user that system is | of pending user | of pending user | of pending user |
| | | user | in use | Notify requesting | Notify requesting | Notify requesting |
| | | Notify | No timeout | user that system is | user that system is | user that system is |
| | | requesting user | Put in queue or | in use | in use | in use |
| | | that system is | callback | Set timeout = 1 m | Set timeout = 5 m | No timeout |
| | | in use | | | Queue or callback | Callback |
| | | Release | | | | |
| | | control | | | | |
| | Service | Warn current | Notify requesting | Warn current user | Warn current user | Not Allowed |
| | | user of pending | user that system is | of request | of pending user | |
| | | user | in use | Notify requesting | Notify requesting | |
| | | Notify | No timeout | user that system is | user that system is | |
| | | requesting user | Callback | in use | in use | |
| | | that system is | | No timeout | No timeout | |
| | | in use | | Callback | Queue or callback | |
| | | No timeout | | | | |

The information transmitted between the station 16 and the robot 12 may be encrypted. Additionally, the user may have to enter a password to enter the system 10. A selected robot is then given an electronic key by the station 16. The robot 12 validates the key and returns another key to the station 16. The keys are used to encrypt information transmitted in the session.

The robot 12 and remote station 16 transmit commands through the broadband network 18. The commands can be generated by the user in a variety of ways. For example, commands to move the robot may be generated by moving the joystick 32. The commands are preferably assembled into packets in accordance with TCP/IP protocol.

The robot controller can operate in accordance with instructions that provide autonomous movement. The autonomous movement may allow the robot to move within a healthcare facility. By way of example, the robot may move from the bed of one patient to the bed of another patient. This allows the robot to automatically perform a patient rounding technique without control by an operator at a remote station. By way of example, the robot may operate software programs referred to as Willow Garage ROS Navigation Stack or MobileRobots Motivity that provide autonomous robot movement.

The infrared robot camera 38B can be used to capture an image of a patient. The image may be processed to determine whether the patient needs assistance. For example, the patient's relative position to a bed can be analyzed to determine the need for assistance. By way of example, if a patient has fallen out of a bed, the robot can determine this from the captured image and then automatically contact a remote station to inform remote medical personnel. The robot can also determine whether the patient may fall out of bed by analyzing the image. Image analysis can be performed with a PrimeSense depth sensor and skeletal mapping software. Likewise, the patient image can be analyzed to determine whether a patient connected to lines and/or restraints, is in an improper position that would require medical assistance.

The robot may contain information regarding various remote control stations so that the proper medical specialist is contacted after analysis of the patient. For example, the infrared image can be analyzed to determine irregular blood flow or excessive body temperature. The robot may automatically contact the remote station of a cardiologist. The robot may generate instructions for the patient to move one or more limbs. The robot may analyze patient movement to determine whether there are any neurological issues and contact a neurologist, accordingly. The patient images can be transmitted to the remote stations for review by the specialist. The images may be archived so that the specialist can see the patient at various time periods.

The robot can be maneuvered through an emergency room or triage area. The robot can be programmed to perform automated analysis that assist in the triage of a patient. The infrared camera allows the robot to capture patients' images through a curtain. This allows for patient diagnosis while maintaining a level of privacy.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A robot system that can move about two or more patient beds, comprising:
a robot that includes;
a mobile platform;
a monitor coupled to said mobile platform;
an infrared camera coupled to said mobile platform; and
a controller that is programmed to autonomously move said mobile platform from one patient to another patient and process images captured by said infrared camera to determine if one or more of the patients needs assistance.

2. The robot system of claim 1, further comprising a remote control station that can control said mobile platform and has a monitor coupled to said infrared camera and a camera coupled to said robot monitor.

3. The robot system of claim 2, wherein said robot initiates communication with said remote station based on a need for a medical specialist.

4. The robot system of claim 1, wherein said controller is programmed to analyze the patient image to determine a position of the patient relative to the patient bed.

5. The robot system of claim 1, wherein said robot generates instructions to be followed by the patient and then analyzes patient movement for a neurological diagnosis.

6. The robot system of claim 1, wherein the patient image is analyzed to determine blood flow of the patient.

7. The robot system of claim 1, wherein said robot moves about an emergency room area.

8. The robot system of claim 7, wherein said infrared camera captures said patient image through a curtain located between said robot and the patient.

9. A method for monitoring two or more patients each located on a patient bed, comprising:
moving autonomously a robot from one patient bed to another patient bed that has a patient, the robot including a monitor and an infrared camera;
capturing at least one infrared image of the patient; and
processing the infrared image to determine whether the patient needs assistance.

10. The method of claim 9, further comprising moving the robot in response to commands from a remote control station and displaying an image of an operator of the remote control station on the robot monitor.

11. The method of claim 10, further comprising initiating communication between the robot and the remote station based on a need for a medical specialist.

12. The method of claim 9, wherein the infrared image is analyzed to determine a position of the patient relative to the patient bed.

13. The method of claim 9, further comprising generating instructions by the robot to be followed by the patient and then analyzing patient movement for a neurological diagnosis.

14. The method of claim 9, wherein the infrared image is analyzed to determine blood flow of the patient.

15. The method of claim 9, wherein the robot moves about an emergency room area.

16. The method of claim 9, wherein the infrared camera captures the infrared image through a curtain located between the robot and the patient.
